(19) United States
(12) Patent Application Publication
(10) Pub. No.: US 2003/0013128 A1
(43) Pub. Date: Jan. 16, 2003

(54) Characterizing nucleic acid and amino acid sequences in silico

(76) Inventors: Arturo J. Morales, Arlington, MA (US); Qiandong Zeng, Belmont, MA (US)

Correspondence Address: BURNS DOANE SWECKER & MATHIS L L P, POST OFFICE BOX 1404, ALEXANDRIA, VA, 22313-1404, US

(21) Appl. No.: 10/175,829

(22) Filed: Jun. 21, 2002

Related U.S. Application Data
(60) Provisional application No. 60/300,586, filed on Jun. 22, 2001.

(51) Int. Cl. G01N 33/53; G01N 33/554; G01N 33/569; G06F 19/00; G01N 33/48; G01N 33/50

(52) U.S. Cl. 435/7.1; 435/7.32; 702/19

(57) ABSTRACT

The invention relates generally to molecular biology and bioinformatics. In particular, the invention related to in silico methods of characterizing nucleic acid and amino acid sequences. In addition, the invention relates to identifying conserved residues and producing an evolutionary profile.

### CROSS-REFERENCE TO RELATED APPLICATION

This application claims priority under 35 U.S.C. §119 to U.S. Provisional Application No. 60/300,586 entitled “Characterizing Nucleic Acid and Amino Acid Sequences In Silico” filed Jun. 22, 2001, the entire content of which is hereby incorporated by reference in its entirety for all purposes.

### FIELD OF THE INVENTION

The invention relates generally to molecular biology and bioinformatics. In particular, the invention relates to in silico methods of characterizing nucleic acid and amino acid sequences. In addition, the invention relates to identifying conserved residues and producing an evolutionary profile.

### BACKGROUND

The interaction between proteins is fundamental to a broad spectrum of biological function including regulation of metabolic pathways, immunological responses, DNA replications, and protein synthesis (Gough et al., Bioinformatics, 17: 455-60 (2001)). Current techniques in elucidating protein-protein interactions and protein functions are tedious and often involved experimental techniques such as the yeast-two-hybrid system. In addition, while efforts from the Human Genome Project and other sequencing efforts continue to identify genes, the function of the genes and resulting proteins is lacking. For example, the budding yeast Sacchromyces cerevisiae was fully sequenced in April 1996 however, one-third of the predicted open reading frames (ORFs) are still classified as unknown function (Uetz et al., Nature, 403: 623-627 (2000)). In contrast to current techniques, the present invention provides the means to identify protein-protein interactions based on primary sequence and structure. In another embodiment, the invention provides a method of identifying the same using solely primary sequence.

### DESCRIPTION OF FIGURES

FIG. 1 depicts the flowchart of the methodology described herein.

FIG. 2 shows a diagram of a system for identifying protein-protein relationships.

FIG. 3 shows a flow diagram describing a method for identifying protein-protein relationships.

FIG. 4 shows the protein relationship of an amino acid biosynthesis protein.

### SUMMARY OF INVENTION

The invention relates to a method of identifying a protein-protein interaction and protein function in silico. Such method includes: i.) compiling a database of sequences; ii.) comparing a reference sequence to at least one sequence in the database; iii.) identifying conserved residues between the reference sequence and at least one sequence in the database sequences; iv.) comparing the conserved residues between the reference sequence and the database sequences; and v.) identifying the protein-protein relationship based on the comparison.

In another embodiment the invention relates to: i.) compiling a database of sequences; ii.) comparing a reference sequence to the database;identifying conserved residues between the reference sequence and the database sequences; iii.) compiling the conserved residues across the reference sequence and the database sequences into a positional vector; iv.) calculating a score for each positional vector; v.) grouping the positional vectors into evolutionary clusters based on the score; vi.) comparing each conserved residue between the reference sequence and database sequences of the evolutionary cluster; vii.) establishing a score at each conserved residue position across the evolutionary cluster; viii.) forming an evolutionary profile based on the scores of the evolutionary clusters; and ix.) based on the evolutionary profile, identifying the protein-protein relationship.

In yet another emobodiment, the invention relates to using the structure the primary sequence to identify the protein-protein interaction and function including: i.) compiling a database of sequences; ii.) comparing a reference sequence to at least one sequence in the database; iii.) identifying conserved residues between the reference sequence and at least one sequence in the database sequences; iv.) compiling conserved residues based on location in structure; v.) forming an evolutionary cluster based on the compiled residues; vi.) comparing each conserved residue between the reference sequence and database sequences of the evolutionary cluster; vii.) establishing a score at each conserved residue position across the evolutionary cluster; viii.) forming an evolutionary profile based on the scores of the evolutionary clusters; and ix.) based on the evolutionary profile, identifying the protein-protein relationship.

### DETAILED DESCRIPTION OF THE INVENTION

Definitions

To aid in the understanding of the specification and claims, the following definitions are provided.

Protein-protein interaction or protein-protein relationship generally refer to at least two proteins that are functionally related which form part of the same or similar biochemical pathway or biological process. The terms also refer to proteins that share similar structure.

Assembled-sequence refers to a sequence composed of at least one non-overlapping segment of sequence. The sequence can comprise, for example, nucleic acid or amino acid sequences.

Conserved Residue refers to a substitution in an amino acid sequence which does not substantially alter the polypeptide's structure and/or activity. These conserved residues are ones which may not be important for protein acitivity or a substitution of an amino acid with a residue having similar properties (acidity, charge, polarity, etc.) such that the substitution may be a critical amino acid but it does not substantially alter the structure and/or activity. Examples of such conserved residues include, but are not limited to Table 1.

TABLE 1

| Original Residue | Conservative Substitution(s) |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Len, Ile |
| Phe | Met, Leu, Tyr |
| Ser | Thr |
| Thr | Ser |
| Trp | Tyr |
| Tyr | Trp, Phe |
| Val | Ile, Leu |

Conserved Bases refer nucleic acid bases which encode for conserved amino acid bases. Conserved Bases also refer to nucleic acid substitutions which do not alter the resulting amino acid sequence. For example, a codon consist of three (3) nucleic acid bases which encode for one (1) amino acid. Due to the degeneracy of the code, one (1) or more of the three (3) nucleic acid bases could be substituted or altered and encode for the same amino acid. For example as in the codons that encode for valine which include GUU, GUA, GUC.

Conserved sequence refers to at least six (6) bases for nucleic acid sequences or two (2) residues for amino acid sequences which are conserved between two (2) or more sequences.

Positional Vector refers to a mathematical description of the conserved residues of the reference sequence and the database sequences. In some instances, the positional vector refers to a matrix that is linearized into one-dimensional vector of length N², where N is the number of sequences in the alignment.

Evolutionary Cluster refers to at least two (2) conserved residues between the reference sequence and the database sequences.

Evolutionary Profile refers to the mathematical description of an evolutionary cluster based on the statistical scoring of conserved residues.

The invention described herein relates to a means of elucidating protein-protein relationships and protein function in silico. One could identify proteins which are essential or proteins which are involved in essential pathway of an organism. This type of information could be used to identify certain drug targets. For example, a protein that is identified as being essential in a bacteria or pathogen could be used in antibiotic screening and discovery. In addition, for instance, an interactor in the inflammatory system of a human could be identified and used in screening agents that prevent inflammatory diseases such as asthma. Additionally, the invention can help target certain active site regions to aid in drug discovery. Other uses include helping group a protein-coding gene into its proper functional unit, and providing 3-D structure validation by showing high homology to proteins of known structure.

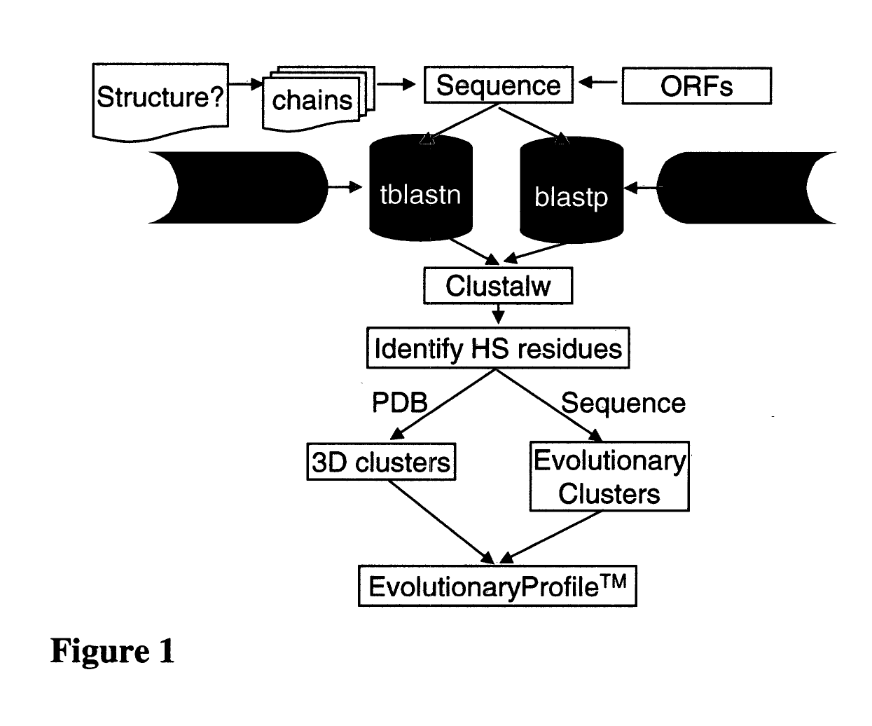

The invention provides a method of compiling nucleic acid and amino acid sequences (See FIG. 1). The compilation could include nucleic acids or amino acid sequences. Preferably, the nucleic acid sequences contains an open reading frames (ORFs). Even more preferably, the sequences include amino acid sequences of the ORFs. The sequences can be derived from eukaryotes, prokaryotes or a combinations thereof. In one embodiment the database contains bacteria sequences. For example the bacteria could be E. coli.

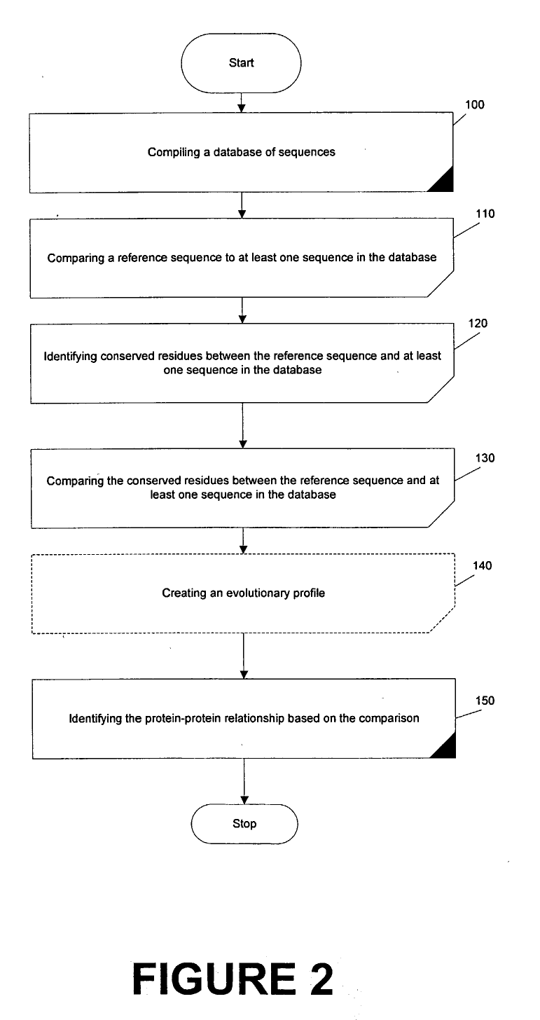

FIG. 2 shows a flow diagram describing a method for identifying protein-protein relationships. In referring to FIG. 2, in step 100, a database containing the structure of proteins can also be created by the following: A subset of the PDB database (Berman, et al., Nucleic Acid Res., 28:235-242 (2000)) containing a set of unique structures with 99% but more prefereably <95% sequence identity is created and those structures separated into individual chains. The sequence identity cutoff used in the creation of the subset database can also be set to 20% but more preferably <30% identity to further lower the redundancy in the dataset.

The reference sequence is the sequence in which the analysis is performed to determine the protein-protein interactions. The reference sequence could be a nucleic acid sequence or an amino acid sequence. The reference sequence could also be combination thereof. Preferably, the reference sequence contains a partial open reading frame or is an expressed sequence tag (EST). More preferably, the sequence contains a full length open reading frame. If the reference sequence is a nucleic acid sequence, the reference sequence would contain at least 10 bases. Alternatively, the reference sequence could be an amino acid sequences, containing at least 5 residues. There may also be more than one reference sequence used in the methodology.

In step 110, in comparing the reference sequence to the database, various algorithms are used including optimal alignment of sequences for comparison can be conducted, e.g., by the local homology algorithm of Smith & Waterman (Adv. Appl. Math., 2:482 (1981)), by the homology alignment algorithm of Needleman & Wunsch (J. Mol. Biol., 48:443 (1970)), by the search for similarity method of Pearson & Lipman (Proc. Natl. Acad. Sci. USA, 85:2444 (1988)) by computerized implementations of these algorithms (CLUSTAL, GAP, BESTFIT, FASTA (Pearson Proc. Tanl. Acad. Sci. USA, 85(8):2444-2448 (1998)) and TFASTA in the Wisconsin Genetics Softare Package, Genteics Computer Group, 575 Science Drive, Madison, Wis.) and BLITZ (Altschul J. Mol. Biol., 215:403-410 (1990)), or by manual alignment and visual inspection.

For example, in comparing a reference sequence which is an amino acid sequence, the algorithm could be BLASTP2. In comparing a reference sequence which is a nucleic acid sequence, BLASTN could be used. In addition, in comparing a reference sequence which is an amino acid sequence, TBLASTN2 could be used against a database of translated nucleotide sequences. In determining the database sequence(s) which contain conserved sequences, a subset of database sequences are chosen based on parameters that one skilled in the art would recognize for such a comparison. For instance in using the the BLAST algorithm, statistical methods can be used to judge the significance of possible matches. The statistical significance of an alignment score is described by the probability, P, of obtaining a higher score when the sequences are shuffled. One way to compute P value threshold is to first consider the total number of sequence comparisons that are to be performed. For example, if there are N proteins in E. coli and M in all other genomes this number is N×M. If a comparison of this number of random sequences would result in one pair to yield a P value of 1/ NM by chance this then is set as the threshold. In the preferred emobodiment, the P-value is <10⁻⁵.

In step 120, in identifying the conserved residues or bases between the database sequences and the reference sequence, additional algorithms are utilized, including but not limited to, Clustal W program (Thompson, Nuc. Acids Res., 22:4673-4680 (1994); Higgins, Methods Enzymol, 266:383-402 (1996)) and PileUp (Devereaux, Nuc. Acids Res., 12:387-395 (1984)). Variations can also be used, such as CLUSTAL X (Jeanmougin, Trends Biochem Sci., 23:403-405 (1998); Thompson, Nucleic Acids Res., 25:4876-4882 (1997)). In the preferred embodiment, the sequences are aligned automatically in a multiple sequence alignment using ClustalW using small gap penalties with the following parameters “-PWGAPOPEN=2.5-GAPOPEN =2.5-PWGAPEXT=0-GAPEXT=0-MAXDIV=20%”. One skilled in the art would appreciate that these parameters can be varied empirically depending on the subset of sequences obtained in the comparison. Every base or residue position from the reference sequence is then scored and compared to all other sequences using an evolutionary scoring matrix such as BLOSUM 62 (Henikoff, Proc. Natl. Acad. Sci. USA, 89:100915 (1989)) or PAM250 and a conservation score for each position is defined as the sum of all scores.

High scoring residues (“conserved residues” for amino acids and “conserved bases” for nucleic acids that encode for conserved residues) are selected and clustered based on structural or evolutionary space as follows: In cases in which the structure of the chain is available, the atoms surrounding the conserved residue or base are investigated further. The distance from the could be 1 Angstrom (Å) or up to 10 Å. More preferably, the distance from the conserved residue or base in between 3 to 7 Å. This area surrounding the conserved residue or base is called the sphere. If there are bases or residues within the sphere which are also conserved, the atoms are grouped together. These residues can then be clustered using an algorithm biased towards surface/exposed clusters by counting all atoms within 1 to 20 Å, more preferable 3 to 7Å from each residue, and concentrating on those residues with fewer atoms around them. This type of clustering identifies important structural motifs where there is some evolutionary pressure to conserve structural and functional characteristics.

When analyzing sequences with no known structures, a positional vector is formed or compiled. In step 130, a matrix of values is calculated using BLOSUM, PAM or Dayhoff algorithm of all possible pairwise comparisons using the evolutionary scoring matrix amongst all species for each high-scoring residue (“conserved residue”) from the original sequence. The matrix is then linearized into N²-dimensional vectors, also known as “positional vectors”, where N is the number of sequences in the alignment, and calculated correlation and euclidean distances amongst all those vectors. Positional vector pairs that have a correlation coefficient of anywhere from 1 to >0.5 and/or were deemed as close in euclidean space are grouped together into “evolutionary clusters.” The exact metric for the euclidean cutoff is determined at runtime with the sole requirement being that the euclidean cutoff is a positive number, to ensure that it is possible to group vectors based on euclidean distance, in addition to correlation. Other distance methods could also be used, such as correlation distance or Manhattan correlation. Initial groups identified in this manner are then merged if they have members in common and their correlation/euclidean distance is above the desired threshold. The merging of these positional vectors into evolutionary clusters can also be achieved using other techniques such as K-means clustering, Self-Organizing maps or Hierarchical Clustering.

In analyzing these evolutionary clusters a pairwise scores are calculated amongst species consisting of the sum of the BLOSUM scores or its equivant for every position in the evolutionary cluster to create a symmetrical N×N matrix. In step 140, this matrix is then linearized using the top half to create or compile a N(N−1)/2 dimensional vector known as an “evolutionary profile”. The evolutionary profile is then normalized to between 0 and 100 with “−100” indicating a missing value. One of ordinary skill in the art would recognize that other normalization methods may be employed as long as they result in a common range for all vectors from a dataset.

This procedure is repeated for every sequence and structure in the dataset. Each evolutionary profile (10-20,000 from an average dataset) is then compared against all other profiles in the dataset and those that have a correlation coefficient of 0.1 or higher, but more preferably 0.5 or higher (or 0.5 or lower) are ranked based on their euclidean distance from the sequence of interest. One skilled in the art would be able to identify other changes and “cutoffs” which could be varied to relax or increase the stringency of the clustering. In addition, other clustering methods such K-means, Hierarchical clustering, Self-Organizing Maps or Principal Component Analysis can be used to analyze the data.

In step 150, to identify the protein-protein relationship of the reference sequence, the evolutionary profiles which result from the ranking using euclidean distances, absolute correlation, Manhattan distance, or other related means, are compiled. The closest “neighbors” based on the compilation of reference sequence's evolutionary profile to the database sequence's evolutionary profiles are then listed on a file and/or written to a database for further analysis and validation.

By examining its closest neighbors, the reference sequence protein-protein interaction can be inferred. In addition, the function and pathways of the reference sequence can also be determined by the compilation. For example, if an ORF has neighbors that are consistently involved in translation, the inference is that it is related to the translation machinery. For more information, see Example 1.

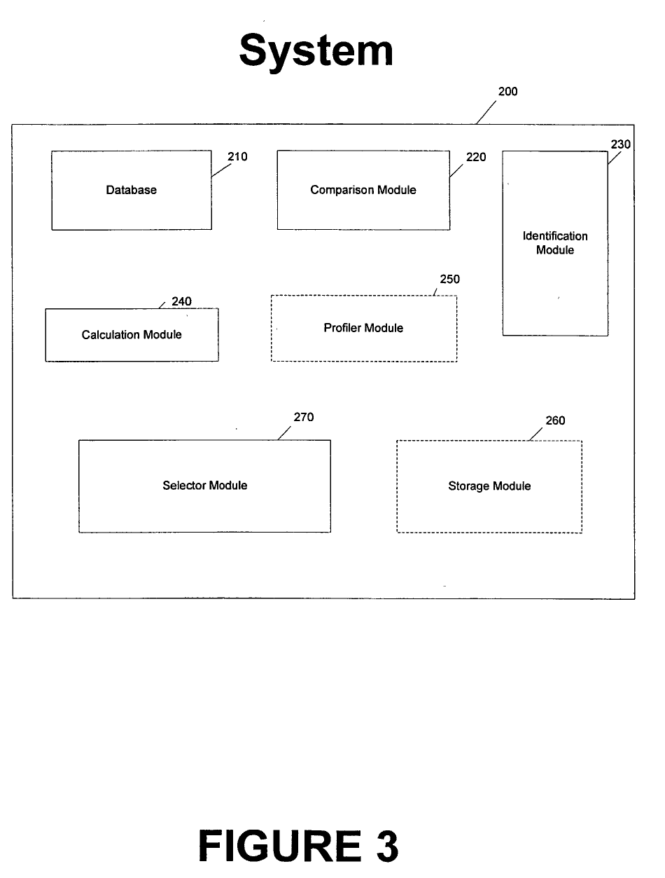

In another embodiment, the invention compiles a database of sequences. Preferably, the database contains sequence information for many different organisms. The reference sequence is compared with the sequences of the database. Segments from the sequences of the database, which closely match the reference sequence, are identified. Preferably, segments are identified using BLAST. Even more preferably, all the non-overlapping segments are identified for each organism in the database. Usually the number of segments identified for an organism depends on the nature of the sequences. For example, if the sequence information of the organism contains introns, non coding sequences, then the BLAST algorithm will return multiple segments for each organism. However, if the sequence information does not contain any introns then only one segment may be identified per organism. The non-overlapping segments are assembled to form an assembled-sequence to be used for analysis. Preferably, one assembled-sequence is created for each organism of the database. The invention identifies the conserved residues between the reference sequence and the assembled-sequences. Subsequently, the conserved residues are compared between the reference sequence and the assembled-sequences. Preferably, an evolutionary profile is created from the comparison. Based on the comparison, protein-protein relationships are identified. Preferably, the protein-protein relationships are identified by comparing evolutionary profiles. FIG. 3 shows a flow diagram describing a method for identifying protein-protein relationships. In referring to FIG. 3, the system 200 includes several modules: a database 210, which contains a plurality of sequences; a comparison module 220, which compares a reference sequence with sequences in the database 210; an identification module 230, which identifies conserved residues shared between the reference sequence and sequences in the database 210; a computational module 240, which computes a value based on the number of conserved residues shared between two sequences; a profiler module 250, which assembles a series of values to form an evolutionary profile, a storage module 260, which stores the evolutionary profile; and a selector module 270, which identifies protein-protein relationships by comparing two evolutionary profiles. Although the system 200 is described to run on a UNIX workstation, the system 200 can be run on other machines including the Macintosh, Windows, Linux, Sun, DOS and others.

A system 200 used for identifying at least one protein-protein relationship will now be described with reference to FIG. 3. The system 200 comprises a database 210 containing a plurality of sequences. The database 210 may include either nucleic acid or amino acid sequences. Preferably, the nucleic acid sequences contain open reading frames (ORFs). Even more preferably, the sequences could include amino acid sequences of the ORFs. The sequences can be derived from eukaryotes, prokaryotes or a combination thereof. The database 210 may contain ORFs from prokayotes and eukayotes. The database 210 may contain ORFs from bacteria. The database 210 may contain ORFs from E. coli.

In the comparison module 220, a reference sequence may be compared with sequences in the database 210 of sequences. Different algorithms may be used to compare the reference sequence with the sequences of the database 210. The comparison module 220 may incorporate different algorithms when analyzing the sequences of the database to find the closest matching sequence. Preferably, sequences of multiple organisms are stored in the database and comparison module 220 finds the closest matching sequence for each organism. For example, if the database 210 contained the entire sequence for 87 different organisms, the comparison module would return a subset containing the 87 closest matching sequences with one matching sequence for each organism. The algorithm used to compare the sequences and identify the closest match could be any one of the following BLAST, FASTA, or its equivalent. The algorithm may weigh sequence matches differently based on the nature of the sequence.

After the subset of the sequences is identified, an identification module 230 identifies conserved residues between the reference sequence and subset of the sequences. Preferably, the identification module 230 identifies only the most highly conserved residues of the subset. More preferably, the residues should not be all weighted equally. The algorithm used to identify the conserved residues includes ClustalW, PileUp or its equivalent. Preferably, the algorithm performs a pair wise comparison between the residues for the members of the subset. Even more preferably, as a result of the pair wise comparisons, the scoring of the residues is calculated using BLOSUM, PAM, Dayhoff, or its equivalent. A table containing the weight of different comparisons may be used to score each pair wise comparison. The conserved residue positions with the highest score beyond a certain cutoff will be saved for further analysis.

Once the conserved residues are identified, the computational module 240 computes a value based on all certain conserved residues shared between the reference sequence and sequences of the subset. The set of conserved residues to be analyzed is called an evolutionary cluster. A reference sequence may contain more than one evolutionary cluster. Based on comparing the evolutionary clusters between two different sequences, a value is computed. Preferably, a value is computed by comparing a sequence with another sequence in the subset of sequences. As a result, the computational module 240 would calculate up to N² values based on N where N is the number of sequences in the subset of sequences. Preferably, N is equivalent to the number of organisms in the database 210 of sequences. Even more preferably, the computational module would create a matrix of N×N values.

A profiler module 250 creates an evolutionary profile grouping together a set of values into a vector. The values that make up the evolutionary profile are based on the calculations of conserved residues of the evolutionary cluster shared between a first sequence of a subset of sequences of the database 210 with a second sequence of the subset. Preferably, the evolutionary profile consists of a vector of values up to a length of N² where N is the number of sequences in the subset. More preferably, assuming the calculations are redundant, the evolutionary profile will consist of values from the top half of the matrix to form a linearized vector of up to N*(N−1)/2 in length.

A storage module 260 stores the evolutionary profile for comparison with other evolutionary profiles. The storage module may reside in RAM, in hard disk, or on another networked computer.

A selector module 270 identifies protein-protein relationships based on a comparison between the evolutionary profile and other evolutionary profiles. The comparison measures the correlation coefficient between the evolutionary profile and the other evolutionary profiles. If the correlation coefficient reaches a cutoff point, for example 0.5, that evolutionary profile is saved. The saved evolutionary profiles are ranked utilizing the Euclidean distance or the Manhattan distance from the evolutionary profile. Based on the Euclidean distance or the Manhattan distance, the reference sequence protein-protein relationship can be inferred.

### EXAMPLE

The example as set forth herein are meant to exemplify the various aspects of the present invention and are not intended to limit the invention in any way.

Following the flowchart in FIG. 1, a database was compiled containing FASTA sequences consisting of all stop-stop open reading frames (ORFs) from sixty-four fully sequenced organisms and all predicted proteins from S. cerevisiae, C. elegans and D.melanogaster was constructed from public and propietary genomes including Genome Therapeutics Corporation PathoGenome™ Database (genomecorp.com) and TIGR's microbial database (tigr.org/tdb/mdb/mdb.html). This resulted in a database consisting of 67 organisms. This database is expected to grow as more complete genomes become available. The current database contains the following species (followed by number of ORFs).

| | |
|---|---|
| A AEOLICUS | 8089 |
| A BAUMANNII | 12038 |
| A FULGIDUS | 13476 |
| A FUMIGATUS | 175346 |
| A PERNIX | 10173 |
| A ANTHRACIS | 18565 |
| B BURGDORFERI | 1253 |
| B FRAGILIS | 26977 |
| B HALODURURANS | 18307 |
| B SP | 1401 |
| B SUBTILIS | 4099 |
| C ACETOBUTYLICUM | 12353 |
| C ALBICANS | 44462 |
| C CRESCENTUS | 35325 |
| C ELEGANS | 18424 |
| C JEJUNI | 5779 |
| C MURIDARUM | 818 |
| C PNEUMONIAE | 1529 |
| C PSITTACI | 1388 |
| C TEPIDUM | 449 |
| C TRACHOMATIS | 887 |
| D ETHENEGENES | 9066 |
| D MELANOGASTER | 18032 |
| D RADIODURANS | 28101 |
| D VULGARIS | 39046 |
| E CLOACAE | 33289 |
| E COLI | 4257 |
| E FAECALIS | 17477 |
| E FAECIUM | 11346 |
| G SULFURREDUCENS | 36705 |
| H INFLUENZA | 1706 |
| H PYLORI | 2994 |
| H SP | 21444 |
| K PNEUMONIAE | 40677 |
| L LACTIS | 7229 |
| M AVIUM | 55417 |
| M CATARRHALIS | 7426 |
| M GENITALIUM | 467 |
| M JANNASCHII | 1714 |
| M LEPRAE | 27491 |
| M LOTI | 68565 |
| M PNEUMONIAE | 677 |
| M THERMOATOTROPHICUM | 1868 |
| M TUBERCULOSIS | 3881 |
| N MENINGIDITIS | 20677 |
| P ABYSSI | 7714 |
| P AERUGINOSA | 60987 |
| P HORIKOSHII | 6762 |
| P MIRABILIS | 11191 |
| P MULTOCIDA | 7738 |
| P PUTIDA | 53095 |
| R PROWAZEKII | 834 |
| S AUREUS | 7108 |
| S CEREVISIAE | 6401 |
| S EPIDERMIDIS | 5949 |
| S PCC | 17839 |
| S PNEUMONIAE | 13560 |
| S PUTREFACIENS | 19096 |
| S PYOGENES | 5558 |
| T ACIDOPHILUM | 9201 |
| T FERROOXIDANS | 26286 |
| T MARITIMA | 12700 |
| T PALLIDU | 1030 |
| T VOLCANIUM | 6548 |
| U UREALYTICUM | 611 |
| V CHOLERA | 3781 |
| X FASTIDIOSA | 18374 |

Using TBLASTN2 as the comparison algorithm, one could then compare the reference sequence against a database of sequences of different organisms. When multiple sequences from an organism have segments that show a similarity to a segment of the reference sequence, one can assemble the non-overlapping segments into a larger sequence to maximize the similarity to the reference sequence. This method is especially beneficial for sequences of organisms that contain introns. In addition, one can then minimize the chance of problems caused by missasembled regions within the sequences. The reference database used in this case contains 85 different genomes from Prokaryotes and Eukaryotes available in the public domain in addition to those included in the Pathogenome™ Database.

The list of species included the following (shown as the first letter of the Genus plus up to the first five characters from the species name:

AAEOLI

ABAUMA

AFULGI

AFUMIG

APERNI

ATHALI

ATUMEF

BANTHR

BBURGD

BFRAGI

BHALOD

BSPAPS

BSUBTI

CACETO

CALBIC

CCRESC

CELEGA

CJEJUN

CMURID

CNEOFO

CPNEUM

CPSITT

CTEPID

CTRACH

DETHEN

DMELAN

DRADIO

DVULGA

ECLOAC

ECOLI_

ECUNIC

EFAECA

EFAECI

GSULFU

HINFLU

HPYLOR

HSAPIE

HSP

KPNEUM

LINNOC

LLACTI

LMONOC

MAVIUM

MCATAR

MGENIT

MJANNA

MLEPRA

MLOTI

MMUSCU

MPNEUM

MPULMO

MTHERM

MTUBER

NCRASS

NMENIN

PABYSS

PAERUG

PFALCI

PHORIK

PMIRAB

PMULTO

PPUTID

RCONOR

RPROWA

SAUREU

SCEREV

SEPIDE

SMELIL

SPCC68

SPNEUM

SPOMBE

SPUTRE

SPYOGE

SSOLFA

STOKOD

STYPHI

TACIDO

TFERRO

TMARIT

TPALLI

TVOLCA

UUREAL

VCHOLE

XFASTI

YPESTI

Tables 2 through 6 show sample results from the methodology described herein. The dataset comprises ˜1500 randomly selected ORFs from E.coli. The ORFs were compared against each other using Evolutionary Profiles and the closest euclidean neighbors for each ORF ranked by distance. Annotation information was extracted from the Kyoto Encyclopedia of Genes and Genomes (KEGG); (Nucleic Acids Res. 28, 29-34 (2000)).

TABLE 2
tufB, factor; Proteins—translation and,
protein chain elongation factor EF-Tu

| | |
|---|---|
| 1. | tufA, factor; Proteins—translation and, protein chain elongation |
| | factor EF-Tu |
| 2. | pyrG, enzyme; Central intermediary metabolism:, CTP synthetase |
| 3. | fliI, enzyme; Surface structures, flagellum-specific ATP synthase |
| 4. | infB, factor; Proteins—translation and, protein chain initiation factor |
| | IF-2 |
| 5. | rplB, structural component; Ribosomal proteins—, 50S ribosomal |
| | subunit protein L2 |
| 6. | hflB, enzyme; Degradation of proteins peptides, sigma32 integral |
| | membrane peptidase |
| 7. | atpA, enzyme; ATP-proton motive force, membrane-bound ATP |
| | synthase F1 sector |
| 8. | thrS, enzyme; Aminoacyl tRNA synthetases tRNA, threonine tRNA |
| | synthetase |
| 9. | lysS, enzyme; Aminoacyl tRNA synthetases tRNA, lysine tRNA |
| | synthetase |
| 10. | lysU, enzyme; Aminoacyl tRNA synthetases tRNA, lysine tRNA |
| | synthetase; heat shock |
| 11. | fusA, factor; Proteins—translation and, GTP-binding protein chain |
| | elongation factor |
| 12. | atpD, enzyme; ATP-proton motive force, membrane-bound ATP |
| | synthase F1 sector |
| 13. | ftsY, membrane; Cell division, cell division membrane protein |
| 14. | eno, enzyme; Energy metabolism carbon: Glycolysis, enolase |
| 15. | rpsK, structural component; Ribosomal proteins—, 30S ribosomal |
| | subunit protein S11 |
| 16. | selB, factor; Proteins—translation and, selenocysteinyl-tRNA-specific |
| | translation |
| 17. | metG, enzyme; Aminoacyl tRNA synthetases tRNA, methionine |
| | tRNA synthetase |
| 18. | lepA, factor; Proteins—translation and, GTP-binding elongation |
| | factor may be inner |
| 19. | ygjD, putative enzyme; Not classified, putative O-sialoglycoprotein |
| | endopeptidase |
| 20. | rpsE, structural component; Ribosomal proteins—, 30S ribosomal |
| | subunit protein S5 |
| 21. | valS, enzyme; Aminoacyl tRNA synthetases tRNA, valine tRNA |
| | synthetase |
| 22. | rpsL, structural component; Ribosomal proteins—, 30S ribosomal |
| | subunit protein S12 |
| 23. | rpoB, enzyme; RNA synthesis modification DNA, RNA polymerase |
| | beta subunit |
| 24. | rplC, structural component; Ribosomal proteins—, 50S ribosomal |
| | subunit protein L3 |
| 25. | aspS, enzyme; Aminoacyl tRNA synthetases tRNA, aspartate tRNA |
| | synthetase |
| 26. | rpoC, enzyme; RNA synthesis modification DNA, RNA polymerase |
| | beta prime subunit |
| 27. | rplM, structural component; Ribosomal proteins—, 50S |

TABLE 3
fliG, structural component; Surface structures,
flagellar biosynthesis component of motor

| | |
|---|---|
| 1. | flgB, structural component; Surface structures, flagellar biosynthesis |
| | cell-proximal portion of |
| 2. | fliC, structural component; Surface structures, flagellar biosynthesis; |
| | flagellin filament |
| 3. | fliG, structural component; Surface structures, flagellar biosynthesis |
| | component of motor |
| 4. | fliN, structural component; Surface structures, flagellar biosynthesis |
| | component of motor |
| 5. | fliM, structural component; Surface structures, flagellar biosynthesis |
| | component of motor |
| 6. | flgE, structural component; Surface structures, flagellar biosynthesis |
| | hook protein |
| 7. | flgF, structural component; Surface structures, flagellar biosynthesis |
| | cell-proximal portion |
| 8. | flgL, structural component; Surface structures, flagellar biosynthesis; |
| | hook-filament junction |
| 9. | flgC, structural component; Surface structures, flagellar biosynthesis |
| | cell-proximal portion of |
| 10. | motA, phenotype; Chemotaxis and mobility, proton conductor |
| | component of motor; no effect |
| 11. | cheA, enzyme; Chemotaxis and mobility, sensory transducer kinase |
| | between chemo-signal |
| 12. | ybiS, orf; Unknown, orf hypothetical protein |
| 13. | fliR, putative enzyme; Surface structures, flagellar biosynthesis |
| 14. | fhiA, putative enzyme; Surface structures, flagellar biosynthesis |
| 15. | ycgB, putative factor; Not classified, putative sporulation protein |
| 16. | ybgA, orf; Unknown, orf hypothetical protein |
| 17. | aer, regulator; Degradation of small molecules:, aerotaxis sensor |
| | receptor flavoprotein |
| 18. | tar, regulator; Chemotaxis and mobility, methyl-accepting chemotaxis |
| | protein II |
| 19. | ynhG, orf; Unknown, orf hypothetical protein |
| 20. | btuB, membrane; Outer membrane constituents, outer membrane |
| | receptor for transport of vitamin |

TABLE 4
rep [DNA-replication repair,
rep helicase, single-stranded DNA dependent]

| | |
|---|---|
| 1. | uvrD, DNA—replication repair, DNA-dependent ATPase I and |
| | helicase II |
| 2. | ruvB, DNA—replication repair, Holliday junction helicase subunit A; |
| | branch |
| 3. | ybeX, putative transport; Not classified, putative transport protein |
| 4. | polA, DNA—replication repair, DNA polymerase |
| | I 3′—5′ polymerase 5′— |
| 5. | mfd, DNA—replication repair, transcription-repair coupling factor; |
| | mutation |
| 6. | murF, Murein sacculus peptidoglycan, D-alanine:D-alanine-adding |
| | enzyme |
| 7. | thdF, Detoxification, GTP-binding protein in thiophene and furan |
| 8. | yhdG, Not classified, putative dehydrogenase |
| 9. | mraY, Murein sacculus peptidoglycan, phospho-N-acetylmuramoyl- |
| | pentapeptide |
| 10. | yqcB, hypothetical protein |
| 11. | sfhB, Not classified, suppressor of ftsH mutation |
| 12. | yceC, hypothetical protein |
| 13. | yjfG, Not classified, putative ligase |
| 14. | yabO, hypothetical protein |
| 15. | ddlA, Murein sacculus peptidoglycan, D-alanine-D-alanine ligase A |
| 16. | murE, Murein sacculus peptidoglycan, meso-diaminopimelate-adding |
| | enzyme |
| 17. | rnc, Degradation of RNA, RNase III ds RNA |
| 18. | gyrB, DNA—replication repair, DNA gyrase subunit B type II |
| | topoisomerase |
| 19. | ddlB, Murein sacculus peptidoglycan, D-alanine-D-alanine ligase B |
| | affects cell |
| 20. | rpoS, Global regulatory functions, RNA polymerase sigma S |
| | (sigma38) factor |
| 21. | dnaX, DNA—replication repair, DNA polymerase III tau and gamma |
| | subunits; DNA |

TABLE 5
trpC, enzyme; Amino acid biosynthesis: Tryptophan,
N-(5-phosphoribosyl)anthranilate isomerase

| | |
|---|---|
| 1. | trpA, enzyme; Amino acid biosynthesis: Tryptophan, tryptophan |
| | synthase alpha protein |
| 2. | trpB, enzyme; Amino acid biosynthesis: Tryptophan, tryptophan |
| | synthase beta protein |
| 3. | trpE, enzyme; Amino acid biosynthesis: Tryptophan, anthranilate |
| | synthase component I |
| 4. | pabB, enzyme; Biosynthesis of cofactors carriers:, p-aminobenzoate |
| | synthetase component I |
| 5. | hisB, enzyme; Amino acid biosynthesis: Histidine, |
| | imidazoleglycerolphosphate dehydratase and |
| 6. | ilvD, enzyme; Amino acid biosynthesis: Isoleucine, dihydroxyacid |
| | dehydratase |
| 7. | hisC, enzyme; Amino acid biosynthesis: Histidine, histidinol- |
| | phosphate aminotransferase |
| 8. | edd, enzyme; Central intermediary metabolism:, 6-phosphogluconate |
| | dehydratase |
| 9. | hisD, enzyme; Amino acid biosynthesis: Histidine, |
| | L-histidinal:NAD+ oxidoreductase |
| 10. | ribH, enzyme; Biosynthesis of cofactors carriers:, riboflavin synthase |
| | beta chain |
| 11. | leuB, enzyme; Amino acid biosynthesis: Leucine, 3-isopropylmalate |
| | dehydrogenase |
| 12. | aroA, enzyme; Amino acid biosynthesis: Chorismate, 5-enolpyruvyl- |
| | shikimate-3-phosphate synthetase |
| 13. | leuD, enzyme; Amino acid biosynthesis: Leucine, isopropylmalate |
| | isomerase subunit |
| 14. | pheA, enzyme; Amino acid biosynthesis: Phenylalanine, chorismate |
| | mutase-P and prephenate dehydratase |
| 15. | argD, enzyme; Amino acid biosynthesis: Arginine, acetylornithine |
| | deltaaminotransferase |
| 16. | goaG, enzyme; Central intermediary metabolism: Pool, 4-amino- |
| | butyrate aminotransferase |
| 17. | ilvC, enzyme; Amino acid biosynthesis: Isoleucine, ketol-acid |
| | reductoisomerase |
| 18. | lysA, enzyme; Amino acid biosynthesis: Lysine, diaminopimelate |
| | decarboxylase |
| 19. | leuA, enzyme; Amino acid biosynthesis: Leucine, 2-isopropylmalate |
| | synthase |
| 20. | leuC, enzyme; Amino acid biosynthesis: Leucine, 3-isopropylmalate |
| | isomerase (dehydratase) |
| 21. | aroE, enzyme; Amino acid biosynthesis: Chorismate, dehydro- |
| | shikimate reductase |
| 22. | glnA, enzyme; Amino acid biosynthesis: Glutamine, glutamine |
| | synthetase |

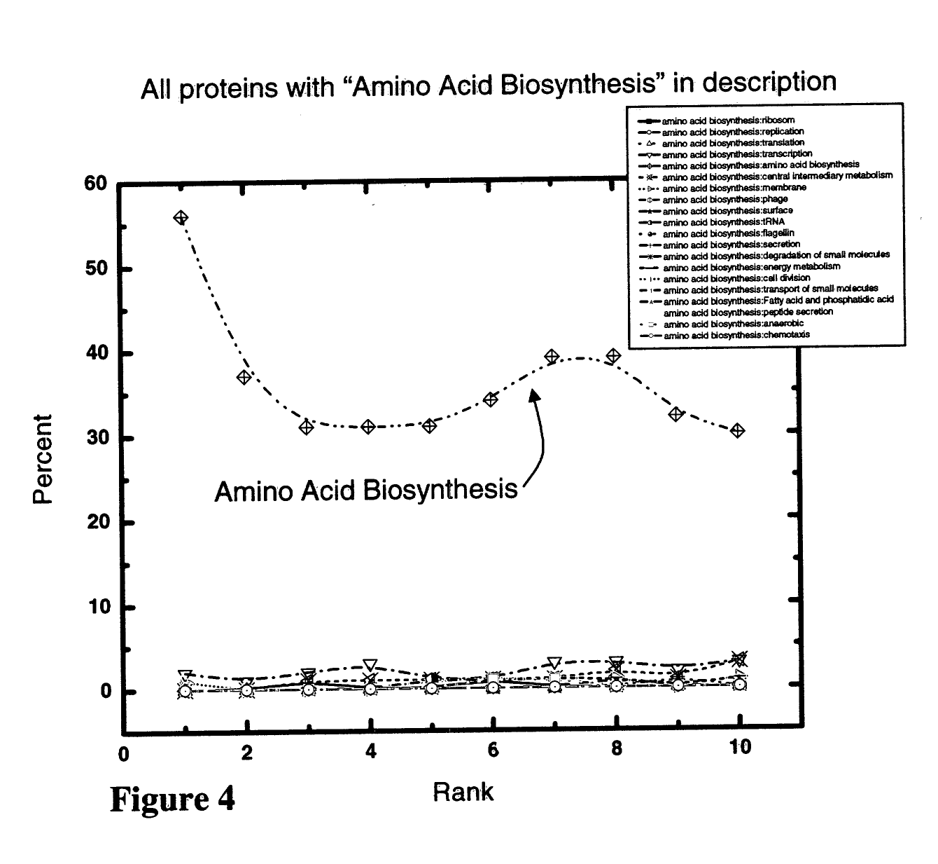

FIG. 4 shows rank percentages for all proteins in the dataset with “Amino Acid Biosynthesis”. The data of FIG. 4 also reflects the information of Table 5. We show the percent occurence of a similar annotation at that rank position based on the methodology described herein. For example, for proteins with “Amino Acid Biosynthesis” in their description, other proteins with the same annotation >60% of the time are related, while none of the other annotations we looked at show up at more than 5% frequency.

TABLE 6
narV, enzyme; Energy metabolism carbon: Anaerobic,
cryptic nitrate reductase 2 gamma subunit

| | |
|---|---|
| 1. | narV, enzyme; Energy metabolism carbon: Anaerobic, cryptic nitrate |
| | reductase 2 gamma subunit |
| 2. | narI, enzyme; Energy metabolism carbon: Anaerobic, nitrate |
| | reductase 1 cytochrome b(NR) gamma |
| 3. | narJ, enzyme; Energy metabolism carbon: Anaerobic, nitrate |
| | reductase 1 delta subunit assembly |
| 4. | narW, enzyme; Energy metabolism carbon: Anaerobic, cryptic nitrate |
| | reductase 2 delta subunit |
| 5. | narZ, enzyme; Energy metabolism carbon: Anaerobic, cryptic nitrate |
| | reductase 2 alpha subunit |
| 6. | narY, enzyme; Energy metabolism carbon: Anaerobic, cryptic nitrate |
| | reductase 2 beta subunit |
| 7. | narH, enzyme; Energy metabolism carbon: Anaerobic, nitrate |
| | reductase 1 beta subunit |
| 8. | narG, enzyme; Energy metabolism carbon: Anaerobic, nitrate |
| | reductase 1 alpha subunit |

Table 7 shows representative results from the method using a dataset comprising about 3,700 Saccaromyces cerevisiae genes processed against the genome database containing 85 genomes. This approach used TBLASTN2 to assemble to non-overlapping high-scoring segments from each organism. This example thus shows the protein-protein relationships which result from the invention described herein.

TABLE 7
RPL11A, Ribosomal subunit/Ribosomal subunit/RNA-binding protein

| | |
|---|---|
| 1. | RPL11B, Ribosomal subunit/RNA-binding protein |
| 2. | RPS9A, /Ribosomal subunit/RNA-binding protein |
| 3. | RPL10, /RNA-binding protein/Ribosomal subunit |
| 4. | RAD51, /DNA-binding protein/ATPase |
| 5. | RPS9B, /Ribosomal subunit/RNA-binding protein |
| 6. | RPL15A, /Ribosomal subunit/RNA-binding protein |
| 7. | SCL1, /Proteasome subunit |
| 8. | DMC1, /ATPase/DNA-binding protein |
| 9. | RPL43B, /RNA-binding protein/Ribosomal subunit |
| 10. | PRE6, /Proteasome subunit/Proteasome subunit |
| 11. | PRE9, /Proteasome subunit |
| 12. | PUP2, /Proteasome subunit |
| 13. | RPL4A, /Ribosomal subunit/RNA-binding protein |
| 14. | RPL4B, /Ribosomal subunit/RNA-binding protein |
| 15. | DYS1, /Oxidoreductase |
| 16. | RPL19B, /Ribosomal subunit/RNA-binding protein |
| 17. | RPS18B, /Ribosomal subunit/Ribosomal subunit/RNA-binding |
| | protein |
| 18. | MCM3, /DNA-binding protein/ATPase/Hydrolase |
| 19. | CDC46, /DNA-binding protein/ATPase/Hydrolase |
| 20. | RPB10, /RNA polymerase subunit |
| 21. | PRE10, /Proteasome subunit/Proteasome subunit |
| 22. | RPO21, /Transferase/RNA polymerase subunit/RNA polymerase |
| | subunit |
| 23. | CDC47, /ATPase/Hydrolase/DNA-binding protein |
| 24. | PRE8, /Proteasome subunit |
| 25. | RPL19A, /Ribosomal subunit/RNA-binding protein |
| 26. | RPL43A, /RNA-binding protein/Ribosomal subunit |
| 27. | RPS18A, /Ribosomal subunit/RNA-binding protein |
| 28. | RPS13, /Ribosomal subunit/RNA-binding protein |

Equivalents

The disclosure of each of the patents, patent applications, and publications cited in the specification is hereby incorporated by reference herein in its entirety for all purposes.

Although the invention has been set forth in detail, one skilled in the art will recognize that numerous changes and modifications can be made, and that such changes and modifications may be made without departing from the spirit and scope of the invention.

We claim:

1. A method of identifying at least one protein-protein relationship comprising:
a. compiling a database of sequences;
b. comparing a reference sequence to at least one sequence in the database;
c. identifying conserved residues between the reference sequence and at least one sequence in the database;
d. comparing the conserved residues between the reference sequence and at least one sequence in the database; and
e. identifying the protein-protein relationship based on the comparison.

2. The method of claim 1, wherein the database contains nucleic acids sequences.

3. The method of claim 1, wherein the database contains amino acid sequences.

4. The method of claim 1, wherein the database contains open reading frame sequences.

5. The method of claim 1, wherein the database contains open reading frame sequences from prokaryotes and eukaryotes.

6. The method of claim 1, wherein the database contains open reading frame sequences from bacteria.

7. The method of claim 1, wherein the database contains open reading frame sequences from E. coli.

8. The method of claim 1, wherein comparing the reference sequence to the database includes the algorithm BLAST, FASTA or its equivalent.

9. The method of claim 1, wherein the identifying of conserved residues includes the algorithm ClustalW, PileUp or its equivalent.

10. The method of claim 1, wherein the identifying of conserved residues includes a pairwise comparison of the reference sequence and the database sequences.

11. The method of claim 10, wherein the identifying of conserved residues further comprises scoring the conserved residues using BLOSUM, PAM, Dayhoff or its equivalent.

12. The method of claim 1, wherein the comparing of conserved residues includes measuring Euclidean distances.

13. The method of claim 1, wherein the comparing of conserved residues includes measuring absolute correlation of the conserved residues.

14. A method of identifying at least one protein-protein relationship comprising:
a. compiling a database of sequences;
b. comparing a reference sequence to at least one sequence in the database;
c. identifying conserved residues between the reference sequence and at least one sequence in the database;
d. comparing the conserved residues between the reference sequence and at least one sequence in the database;
e. grouping the conserved residues; and
f. identifying the protein-protein relationship based on the grouping.

15. The method of claim 14, wherein the database contains nucleic acids sequences.

16. The method of claim 14, wherein the database contains amino acid sequences.

17. The method of claim 14, wherein the database contains open reading frame sequences.

18. The method of claim 14, wherein the database contains open reading frame sequences from prokaryotes and eukaryotes.

19. The method of claim 14, wherein the database contains open reading frame sequences from bacteria.

20. The method of claim 14, wherein the database contains open reading frame sequences from E. coli.

21. The method of claim 14, wherein comparing the reference sequence to the database includes the algorithm BLAST, FASTA or its equivalent.

22. The method of claim 14, wherein the identifying of conserved residues includes the algorithm ClustalW, PileUp or its equivalent.

23. The method of claim 14, wherein the identifying of conserved residues includes a pairwise comparison of the reference sequence and the database sequences.

24. The method of claim 23, wherein the identifying of conserved residues further comprises scoring the residues using BLOSUM, PAM, Dayhoff or its equivalent

25. The method of claim 14, wherein the comparing of conserved residues includes measuring Euclidean distances.

26. The method of claim 14, wherein the comparing of conserved residues includes measuring absolute correlation of the conserved residues.

27. The method of claim 14, wherein the grouping includes combining based on Euclidean distance and absolute correlation measurements of the conserved bases.

28. A method of identifying at least one protein-protein relationship comprising:
a. compiling a database of sequences;
b. comparing a reference sequence to at least one sequence in the database;
c. identifying conserved residues between the reference sequence and at least one sequence in the database;
d. forming a positional vector containing the conserved residues;
e. grouping the positional vectors into evolutionary clusters;
f. compiling an evolutionary profile based on the evolutionary clusters; and
g. identifying the protein-protein relationship based on the evolutionary profiles.

29. The method of claim 28, wherein the database contains nucleic acids sequences.

30. The method of claim 28, wherein the database contains amino acid sequences.

31. The method of claim 28, wherein the database contains open reading frame sequences.

32. The method of claim 28, wherein the database contains open reading frame sequences from prokaryotes and eukaryotes.

33. The method of claim 28, wherein the database contains open reading frame sequences from bacteria.

34. The method of claim 28, wherein the database contains open reading frame sequences from E. coli.

35. The method of claim 28, wherein comparing the reference sequence to the database includes the algorithm BLAST, FASTA or its equivalent.

36. The method of claim 28, wherein the identifying of conserved residues includes the algorithm ClustalW, PileUp or its equivalent.

37. The method of claim 28, wherein the identifying of conserved residues includes a pairwise comparison of the reference sequence and the database sequence.

38. The method of claim 37, wherein the identifying of conserved residues further comprises a scoring the residues using BLOSUM, PAM, Dayhoff or its equivalent.

39. The method of claim 28, wherein the forming of positional vectors includes compiling conserved residues at each position within the reference sequence.

40. The method of claim 28, wherein the grouping of positional vectors includes measuring Euclidean distances.

41. The method of claim 28, wherein the grouping of positional vectors includes measuring absolute correlation of conserved residues.

42. The method of claim 28, wherein the grouping includes combining positional vectors based on Euclidean distances and absolute correlation of conserved residues.

43. The method of claim 28, wherein the compiling of evolutionary profiles includes a pairwise comparison of each position of the evolutionary cluster.

44. The method of claim 43, further comprising using the algorithm BLOSUM, PAM, Dayhoff or its equivalent

45. A method of identifying at least one protein-protein relationship comprising:
a. compiling a database of sequences;
b. comparing a reference sequence to at least one sequence in the database;
c. identifying conserved residues between the reference sequence and at least one sequence in the database;
d. compiling the conserved residues across the reference sequence and the database sequences into a positional vector;
e. calculating a score for each positional vector;
f. grouping the positional vectors into evolutionary clusters based on the score;
g. comparing each conserved residue between the reference sequence and at least one sequence in database of the evolutionary cluster;
h. forming an evolutionary profile based on the evolutionary clusters; and
i. based on comparing each evolutionary profile, identifying the protein-protein relationship.

46. The method of claim 45, wherein the database contains nucleic acids sequences.

47. The method of claim 45, wherein the database contains amino acid sequences.

48. The method of claim 45, wherein the database contains open reading frame sequences.

49. The method of claim 45, wherein the database contains open reading frame sequences from prokaryotes and eukaryotes.

50. The method of claim 45, wherein the database contains open reading frame sequences from bacteria.

51. The method of claim 45, wherein the database contains open reading frame sequences from E. coli.

52. The method of claim 45, wherein comparing the reference sequence to the database includes the algorithm BLAST, FASTA or its equivalent.

53. The method of claim 45, wherein the identifying of conserved residues includes the algorithm ClustalW, PileUp or its equivalent.

54. The method of claim 45, wherein calculating the score for each positional vector includes a pairwise comparison of the reference sequence and the database sequences.

55. The method of claim 54, wherein calculating the score for each positional vector further comprising comparing conserved residues using BLOSUM, PAM, Dayhoff or its equivalent.

56. The method of claim 45, wherein the grouping of positional vectors includes measuring Euclidean distances.

57. The method of claim 45, wherein the grouping of positional vectors includes measuring absolute correlation of the conserved residues.

58. The method of claim 45, wherein the grouping includes combining the positional vectors based on Euclidean distance and absolute correlation of the conserved residues.

59. The method of claim 45, wherein the comparing of conserved residues includes a pairwise comparison of each residue at each position of the evolutionary cluster whereby each database sequence and reference sequence is compared to each other.

60. The method of claim 59, further comprising using the algorithm BLOSUM, PAM, Dayhoff or its equivalent.

61. A method of identifying at least one protein-protein relationship comprising:
a. compiling a database of sequences;
b. comparing a reference sequence to at least one sequence in the database;
c. identifying conserved residues between the reference sequence and at least one sequence in the database;
d. compiling the conserved residues across the reference sequence and at least one sequence in the database into a positional vector;
e. calculating a score for each positional vector;
f. grouping the positional vectors into evolutionary clusters based on the score;
g. comparing each conserved residue between the reference sequence and at least one sequence in database of the evolutionary cluster;
h. establishing a score at each conserved residue position across the evolutionary cluster;
i. forming an evolutionary profile based on the scores of the evolutionary clusters; and
j. based on the evolutionary profile, identifying the protein-protein relationship.

62. The method of claim 61, wherein the database contains nucleic acids sequences.

63. The method of claim 61, wherein the database contains amino acid sequences.

64. The method of claim 61, wherein the database contains open reading frame sequences.

65. The method of claim 61, wherein the database contains open reading frame sequences from prokaryotes and eukaryotes.

66. The method of claim 61, wherein the database contains open reading frame sequences from bacteria.

67. The method of claim 61, wherein the database contains open reading frame sequences from E. coli.

68. The method of claim 61, wherein comparing the reference sequence to the database includes the algorithm BLAST, FASTA or its equivalent.

69. The method of claim 61, wherein the identifying of conserved residues includes the algorithm ClustalW, PileUp or its equivalent.

70. The method of claim 61, wherein calculating the score for each positional vector includes a pairwise comparison of the reference sequence and the database sequences.

71. The method of claim 61, wherein calculating the score for each positional vector further comprising comparing conserved residues using BLOSUM, PAM, Dayhoff or its equivalent.

72. The method of claim 61, wherein the grouping of positional vectors includes measuring Euclidean distances.

73. The method of claim 61, wherein the grouping of positional vectors includes measuring absolute correlation of the conserved residues.

74. The method of claim 61, wherein the grouping includes combining the positional vectors based on Euclidean distance and absolute correlation of the conserved residues.

75. The method of claim 61, wherein the comparing of conserved residues includes a pairwise comparison of each residue at each position of the evolutionary cluster whereby each database sequence and reference sequence is compared to each other.

76. The method of claim 75, further comprising using the algorithm BLOSUM, PAM, Dayhoff or its equivalent.

77. A method of identifying at least one protein-protein relationship comprising:
a) compiling a database of sequences;
b) comparing a reference sequence to at least one sequence in the database;
c) identifying conserved residues between the reference sequence and at least one sequence in the database;
d) compiling conserved residues based on location in structure;
e) forming an evolutionary cluster based on the compiled residues;
f) comparing each conserved residue between the reference sequence and at least one sequence in the database of the evolutionary cluster;
g) establishing a score at each conserved residue position across the evolutionary cluster;
h) forming an evolutionary profile based on the scores of the evolutionary clusters; and
i) based on the evolutionary profile, identifying the protein-protein relationship.

78. The method of claim 77, wherein the database contains nucleic acids sequences.

79. The method of claim 77, wherein the database contains amino acid sequences.

80. The method of claim 77, wherein the database contains open reading frame sequences.

81. The method of claim 77, wherein the database contains open reading frame sequences from prokaryotes and eukaryotes.

82. The method of claim 77, wherein the database contains open reading frame sequences from bacteria.

83. The method of claim 77, wherein the database contains open reading frame sequences from E. coli.

84. The method of claim 77, wherein comparing the reference sequence to the database includes the algorithm BLAST, FASTA or its equivalent.

85. The method of claim 77, wherein the identifying of conserved residues includes the algorithm ClustalW, PileUp or its equivalent.

86. The method of claim 77, wherein the compiling of conserved residues is based on the location between the conserved residues measured in Ångstroms.

87. The method in claim 86, wherein the location distance between residues is 3 to 7 Ångstroms.

88. The method of claim 77, wherein the comparing of conserved residues includes a pairwise comparison of each residue at each position of the evolutionary cluster whereby each database sequence and reference sequence is compared to each other.

89. The method of claim 88, further comprising using the algorithm BLOSUM, PAM, Dayhoff or its equivalent.

90. A method of identifying at least one protein-protein relationship comprising:
a. compiling a database of sequences;
b. comparing a reference sequence to at least one sequence in the database;
c. identifying at least one segment of a sequence within a set of sequences of the database;
d. assembling a set of segments to create an assembled-sequence;
e. identifying conserved residues between the reference sequence and at least one assembled-sequence in a set of assembled-sequences;
f. comparing the conserved residues between the reference sequence and at least one assembled-sequence in the set of assembled-sequences; and
g. identifying the protein-protein relationship based on the comparison.

91. A system of identifying at least one protein-protein relationship comprising:
a database of a plurality of sequences;
a Comparison module used to compare a reference sequence to at least one sequence in the database of sequences;
an Identification module to identify conserved residues between the reference sequence and at least one sequence in the database of sequences;
a Calculation module used to compare the conserved residues between the reference sequence and at least one sequence in the database of sequences; and
an Selector module to identify the protein-protein relationship based on the comparison.

92. The system of claim 91, wherein the database contains nucleic acids sequences.

93. The system of claim 91, wherein the database contains amino acid sequences.

94. The system of claim 91, wherein the database contains open reading frame sequences.

95. The system of claim 91, wherein the database contains open reading frame sequences from prokaryotes and eukaryotes.

96. The system of claim 91, wherein the database contains open reading frame sequences from bacteria.

97. The system of claim 91, wherein the database contains open reading frame sequences from E. coli.

98. The system of claim 91, wherein the comparison module further comprises using the algorithm BLAST, FASTA or its equivalent.

99. The system of claim 91, wherein the identification module further comprises identifying of conserved residues using the algorithm ClustalW, PileUp or its equivalent.

100. The system of claim 91 further comprising a profiler module to calculate an evolutionary profile.

101. The system of claim 91 further comprising a storage module to store evolutionary profiles.

102. A computer readable medium, which when executed by a microprocessor, performs a method of identifying at least one protein-protein relationship comprising:
a. compiling a database of sequences;
b. comparing a reference sequence to at least one sequence in the database;
c. identifying conserved residues between the reference sequence and at least one sequence in the database;
d. comparing the conserved residues between the reference sequence and at least one sequence in the database; and
e. identifying the protein-protein relationship based on the comparison.
